(19) United States
(12) Patent Application Publication
(10) Pub. No.: US 2004/0062794 A1
(43) Pub. Date: Apr. 1, 2004

(54) 17Beta- estradiol/levonorgestrel transdermal patch for hormone replacement therapy

(76) Inventors: Lee Shulman, Chicago, IL (US); Kerstin Uhl, Berlin (DE); Vladimir Yankov, Montville, NJ (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/259,892

(22) Filed: Sep. 30, 2002

(51) Int. Cl. A61K 31/56; A61K 9/70

(52) U.S. Cl. 424/449; 514/170

(57) ABSTRACT

The present invention relates to a method for reducing triglyceride levels in a patient and effecting hormone replacement therapy comprising continuously and transdermally administering an essentially constant therapeutically effective amount of a composition comprising an estradiol (17β-estradiol) and a progestin (levonorgestrel (LNG)) in a pharmaceutically acceptable carrier.

### SUMMARY OF THE INVENTION

The present invention relates to a composition for hormone replacement therapy comprising an estrogen and a progestin in a pharmaceutically acceptable transdermal carrier. Further, the present invention relates to a method for reducing triglyceride levels in a patient undergoing hormone replacement therapy comprising administering to a patient, in need thereof, a therapeutically effective amount of said composition.

It has been discovered that the continuous transdermal administration of a composition comprising 17β-estradiol and levonorgestrel (LNG) in therapeutically effective concentrations of both hormones to relieve the symptoms of menopause in women, significantly reduces the level of triglycerides, which are a major risk factor for cardiovascular disease in women, and some lipoproteins.

Hormone replacement therapy (HRT) has long been provided to menopausal and post-menopausal women to relieve menopausal symptoms such as hot flashes, night sweats, calcium loss from bone, and for the prevention of heart disease. These therapies usually involve administering over a time period varying amounts of hormone preparations (estrogens with or without progestins) cyclically, continuously or sequentially to a woman in need of such treatment. Menopause has also been associated with adverse changes to lipid and lipoprotein levels, some of which are important risk factors for coronary heart disease (CHD). These adverse changes include increases in total cholesterol (TC), low-density lipoproteins (LDL), and triglycerides, coupled with slight decreases in high-density lipoproteins (HDL). Many currently available combination HRT therapies have been associated with significant increases in triglyceride levels. Elevated triglycerides, in addition to being a risk factor for CHD in women, has also been associated with insulin resistance and polycystic ovarian disease.

Thus, one aspect of the present invention is a transdermal composition comprising 17β-estradiol and LNG.

Another aspect of the present invention is a method for continuous transdermal hormone replacement therapy for treating the vasomotor and urogenital symptoms of menopausal women. The method comprises continuously administering constant therapeutically effective amounts of 17β-estradiol and LNG in a pharmaceutically acceptable carrier.

Another aspect of the present invention is a method for reducing serum triglyceride levels in a patient undergoing hormone replacement therapy. The method comprises continuously administering essentially constant therapeutically effective amounts of 17β-estradiol and LNG in a pharmaceutically acceptable carrier.

In another aspect, the present invention relates to a transdermal delivery system for the administration of 17β-estradiol and LNG in a pharmaceutically acceptable carrier.

The pharmaceutically acceptable carriers in which the estradiol and progestin are dissolved or suspended include, but are not limited to, aqueous and non-aqueous carriers. The composition of the invention may contain other ingredients/additives. For example, the composition of the invention may contain additional ingredients/additives that may increase the solubility of the active agents in the composition, increase the release of the active agents in the composition, facilitate or enhance the penetration of the active agents in the composition (e.g., isopropyl myristate and glyceryl monolaurate), prevent crystallization of the active agents in the composition, or any other ingredients/additives employed in a composition for the transdermal delivery of a drug composition (e.g., any liquid, gel, solvent, diluent, solubilizer, or the like). It is well within the knowledge of the skilled artisan to select the appropriate additional ingredients/additives and the amounts thereof to include in the composition of the invention.

The preferred delivery route for the composition of the invention is transdermal administration via any of the known transdermal drug delivery systems known in the art. Thus, the composition of the invention may be formulated as a gel, a viscous liquid, an ointment, a cream, or in any other formulation suitable for transdermal application.

In a preferred embodiment, the 17β-estradiol and LNG composition is formulated as a gel for transdermal administration by methods known in the art. More preferably, the composition is delivered transdermally via a patch. There are a number of transdermal patches available commercially which may be used with the composition of the invention, and it is well within the knowledge of the skilled artisan to select the appropriate patch and methods for preparing such a patch. A suitable patch is disclosed in U.S. Pat. No. 6,086,911. Other patches for transdermal delivery of a drug(s) are described in U.S. Pat. Nos. 6,132,760; 6,312,715; 6,193,996; and 6,136,807.

For example, one patch formulation comprises a flexible backing layer; an adhesive coating layer on said backing comprising a polymer (or copolymer), at least one penetration enhancer, at least one organic solvent, 17β-estradiol and LNG; and a protective liner attached to the adhesive. The patch may optionally contain at least one additional layer comprising other ingredients.

In one embodiment, the 17β-estradiol and LNG are preferably administered as a composition formulated for transdermal delivery via a patch. In another embodiment, the 17β-estradiol and the LNG are each formulated for transdermal delivery as separate patches, however, the two 17β-estradiol and LNG patches are administered or applied to the patient simultaneously.

The preferred doses of 17β-estradiol and LNG for transdermal delivery to a patient range from about 3 mg to about 6 mg, more preferably from about 4 mg to about 5 mg, and most preferably from about 4.4 mg to about 4.5 mg of 17β-estradiol and from about 1 mg to about 5 mg, more preferably from about 1 mg to about 4 mg, and most preferably from about 1.39 mg to about 3.75 mg of LNG.

The delivery rates for 17β-estradiol and LNG range from about 0.025 mg/day to about 0.1 mg/day and from about 0.015 mg/day to about 0.040 mg/day, respectively. The preferred doses of 17β-estradiol and LNG are such that any one of the following approximate delivery rates (mg/day) is achieved: about 0.045 mg/day 17β-estradiol and about 0.015 mg/day LNG; about 0.045 mg/day 17β-estradiol and about 0.030 mg/day LNG; and about 0.045 mg/day 17β-estradiol and about 0.040 mg/day LNG. The most preferred delivery rate is about 0.045 mg/day 17β-estradiol and about 0.030 mg/day LNG.

In a preferred embodiment, the 17β-estradiol and LNG patches are administered transdermally to a patient in need thereof once per week for as long as such treatment is desired.

### BRIEF DESCRIPTION OF THE DRAWINGS

FIG. 1: FIG. 1 depicts the change from baseline in the mean weekly hot flush frequency (Study 1).

FIG. 2: FIG. 2 depicts the change from baseline in mean daily hot flush severity (averaged over all treatment weeks in a cycle) (Study 1). Hot flush severity was assessed on a 4-point scale (0=none; 1=mild; 2=moderate; 3=severe).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLE

Safety and Efficacy of a Continuous Once-a-Week 17β-estradiol/levonorgestrel Transdermal System and its Effects on Vasomotor Symptoms and Endometrial Safety in Post Menopausal Women

Study Design

Two prospective multicenter, double-blind, randomized, controlled trials were conducted to examine the safety and efficacy of continuous administration of a combined once-a-week transdermal E₂/LNG delivery system (Berlex Laboratories, Wayne, N.J., USA) on the vasomotor symptoms and endometrium safety in postmenopausal women. Institutional review boards at each study site approved trial protocols and consent procedures. The trials are referred to as study 1 and study 2.

Participants

Inclusion criteria for both trials were as follows: age ≧45 years; amenorrhea for ≧12 months or, if <12 months but ≧6 months, serum E₂ levels <20 pg/mL and follicle-stimulating hormone levels >40 mIU/mL for ≧6 months; in women with an intact uterus, a negative endometrial biopsy or endometrial thickness <5 mm on transvaginal ultrasound (if inadequate tissue); and a negative pregnancy test, if relevant. Women with abnormal Papanicolaou (Pap) smears, suspected malignant or premalignant disease, or any severe chronic condition or condition that would preclude estrogen therapy were excluded. Hormonal therapy (oral, transdermal, intrauterine, intravaginal, depot) was discontinued ≧8 weeks before the start of both trials, and intramuscular hormonal therapy was discontinued ≧6 months before. Informed consent was obtained from each subject before study entry.

Study 1

Symptomatic hysterectomized and nonhysterectomized postmenopausal women from 32 United States centers who experienced seven moderate to severe hot flushes per day for 1 week or ≧60 moderate to severe hot flushes in 1 week during a 4-week run-in period were eligible for inclusion. Hot flush severity was defined as severe (sensation of heat with perspiration causing the subject to stop activity or awaken from sleep), moderate (sensation of heat with perspiration that did not interfere with activity), mild (sensation of heat without perspiration), or none.

Study 2

Postmenopausal women with intact uteri recruited from 73 United States centers, with or without menopausal symptoms, were eligible for inclusion. Women with ≧15 hot flushes (any severity) during any 7 consecutive days of a 2-week run-in period were enrolled in a vasomotor symptom substudy.

Treatment

In both trials, women were randomly assigned to one treatment using a computer-generated code (permuted block method). Randomization was done by center and balanced by block. All study personnel and trial participants were blinded to randomization codes and treatment assignments.

Study 1

Women (n=293) were randomized to transdermal E₂/LNG 4.4/2.75 mg (22 cm²), 4.5/3.75 mg (30 cm²), or placebo patches administered for 12 weeks (three 4-week cycles).

Study 2

Women (n=845) were randomized to transdermal E₂/LNG 4.4/1.39 mg (22 cm²), 4.4/2.75 mg (22 cm²), 4.5/3.75 mg (30 cm²), or unopposed E₂ 4.4 mg (22 cm²) patches administered continuously for approximately 1 year (13×28-day cycles).

Matching placebo patches (22 or 30 cm²) were used in both trials to preserve blindness (double-dummy design). Therefore, each patient applied two patches simultaneously to skin on the abdomen. Patches were worn continuously for 7 days and were changed weekly. Women were required to return unused medication and empty cycle packs at each clinic visit for the purpose of measuring compliance.

Assessment

Study 1

After initial screening, further assessments were performed at baseline and at the end of cycles 1 and 3. The frequency and severity of hot flushes were recorded daily, and urogenital symptoms were recorded weekly throughout the three treatment cycles using an interactive voice response system. Physical and pelvic examinations, Pap smears, and laboratory tests (blood chemistry, hematology, urinalysis, and lipids) were performed at screening and cycle 3, whereas vital signs (blood pressure, heart rate, and weight) were assessed after each cycle. Spontaneously reported adverse events also were recorded throughout the study.

Study 2

After screening and baseline assessments, visits to the clinic were made after the end of cycles 1, 3, 7, 10, and 13. Transvaginal ultrasound was performed at screening and at the end of cycle 7 in all patients; transvaginal ultrasound or endometrial biopsies (in women with an endometrial thickness of ≧5 mm) were performed at the end of cycle 13 (or final visit).

All patients kept a worksheet on which they recorded bleeding and spotting patterns daily for the duration of the study. In the patient subgroup in which symptoms were assessed, the number and severity of hot flushes were recorded daily for the first three cycles, and urogenital symptoms were recorded weekly for the duration of the study. All were recorded using an interactive voice response system.

Quality of life was assessed using the Short Form-36 (physical functioning and mental health domains; data not presented) and the Women's Health Questionnaire (WHQ). Questionnaires were administered at baseline and at the end of cycles 3, 7, and 13 (or final visit). The WHQ examines 36 psychological and somatic symptoms (organized into nine domains) experienced by women and has well-documented reliability and validity (Wiklund et al., Maturitas, 14:225-36 (1992)).

Adverse events, vital signs, and body weight were assessed at the end of cycles 1, 3, 7, 10, and 13. Laboratory tests (blood chemistry, hematology, urinalysis, and lipids), physical examination, and mammograms were performed after cycles 7 and/or 13, and a Pap smear was performed after cycle 13.

Outcome Measures

Study 1

The primary efficacy variable was the change from baseline in the mean weekly number of hot flushes per cycle (4 consecutive weeks). Secondary measures were the change from baseline in the mean daily number of hot flushes, weekly hot flush frequency, and the mean daily maximal severity of hot flush scores according to a four-point scale (0=none; 1=mild; 2=moderate; 3=severe). The proportion of women with urogenital symptoms (vaginal dryness, dyspareunia, frequent urination, dysuria, stress incontinence, nocturia) also was assessed.

Study 2

The primary efficacy measure was the incidence of endometrial hyperplasia or cancer. Secondary measures were changes from baseline in endometrial morphology, mean daily and weekly number of hot flushes, weekly hot flush frequency, mean daily maximal severity of hot flushes, and total/subscores of the WHQ. Other secondary measures were the proportion of women with amenorrhea, the number of bleeding or spotting days, and the proportion of women with urogenital symptoms (vaginal dryness, dyspareunia, polyuria, dysuria, stress incontinence, nocturia).

Statistical Methods

All safety and efficacy variables were assessed using the intent-to-treat (ITT) population, defined as all women randomized to the study and known to have received at least one dose of the study drug. Endpoint analyses, defined as data from the final visit on study medication carried forward, also are presented for all variables, except for the amenorrhea data in study 2, which were presented as a cumulative analysis of completers.

Continuous variables were analyzed by a two-way analysis of variance model, with treatment and pooled center as terms in the model. With one exception, categorical variables were analyzed using the generalized Cochran-Mantel-Haenszel test, adjusted for pooled center. P values for comparisons between treatment groups were adjusted by the Bonferroni procedure and were tested at the significance level of 0.05/2=0.025. A life-table method was used to analyze the incidence of endometrial hyperplasia, and the Fisher exact test was used to compare treatments for this endpoint.

Study 1

A sample size of approximately 300 was required to have the 240 (80/group) completers necessary to detect a between-group difference at a significance level of 0.025 (Bonferroni corrected) with a power of 80%.

Study 2

Assuming a discontinuation rate of approximately 25%, an estimated sample size of 800 subjects was needed for 600 women (150/group) to complete 13 cycles of treatment. This sample size allowed a difference between treatment groups to be detected in the primary endpoint at a significance level of 0.0167 (Bonferroni corrected) with a power of 99% and also allowed for estimation of a dose-response relationship, if any.

Results

The characteristics and demographic details of women in studies 1 and 2 are presented in Tables 1 and 2, respectively. With the exception of smoking history in study 1, there were no significant differences between treatment groups at the time of randomization.

TABLE 1
Summary of baseline characteristics: study 1

| | E₂/LNG mg/day | | | | | | |
|---|---|---|---|---|---|---|---|
| Variable [mean (range)] | 0.045/0.030 | | 0.045/0.040 | | Placebo | | p value |
| Number of subjects | 96 | | 104 | | 93 | | |
| Age (y) | 52.4 | (45-66) | 51.9 | (44-68) | 51.8 | (43-66) | 0.699 |
| Race | | | | | | | |
| Caucasian | 76 | (79.2%) | 83 | (79.8%) | 77 | (82.8%) | 0.912 |
| Black | 14 | (14.6%) | 14 | (13.5%) | 11 | (11.8%) | |
| Hispanic | 5 | (5.2%) | 4 | (3.8%) | 4 | (4.3%) | |
| Asian | 0 | | 1 | (1.0%) | 1 | (1.1%) | |
| Other | 1 | (1.0%) | 2 | (1.9%) | 0 | | |
| Weight (lbs) | 165.0 | (107-250) | 169.3 | (94-325) | 165.7 | (105-346) | 0.633 |
| Smoking history | | | | | | | |
| No | 61 | (63.5%) | 76 | (73.1%) | 74 | (79.6%) | 0.042 |
| Yes | 35 | (36.5%) | 28 | (26.9%) | 19 | (20.4%) | |
| Estradiol (pg/mL) | 6.93 | (1.6-19.8) | 7.27 | (1.5-23.3) | 7.23 | (1.7-33.0) | 0.780 |
| FSH (mIU/mL) | 81.2 | (38-159) | 78.7 | (38-146) | 80.2 | (51-135) | 0.741 |

E₂, 17β-estradiol,
LNG, levonorgestrel,
FSH, follicle-stimulating hormone.

TABLE 2
Summary of baseline characteristics study 2

| Variable | E₂/LNG mg/day | | | | |
|---|---|---|---|---|---|
| [mean (range)] | 0.045/0.015 | 0.045/0.030 | 0 045/0 040 | 0 045/0 0 | p value |
| Number of subjects | 212 | 211 | 213 | 204 | |
| Age (y) | 55.9 (45-75) | 55 9 (44-75) | 55.4 (44-73) | 55 8 (44-76) | 0 817 |
| Race | | | | | |
| Caucasian | 188 (88.7%) | 191 (90 5%) | 196 (92.0%) | 187 (91 7%) | 0.737 |
| Black | 12 (5.7%) | 8 (3.8%) | 10 (4.7%) | 7 (3 4%) | |
| Hispanic | 6 (2.8%) | 9 (4.3%) | 7 (3.3%) | 7 (3 4%) | |
| Asian | 4 (1.9%) | 2 (0.9%) | 0 | 2 (1.0%) | |
| Other | 2 (0.9%) | 1 (0.5%) | 0 | 1 (0.5%) | |
| Weight (lbs) | 161.7 (92-289) | 163.4 (94-276) | 161.9 (95-261) | 163 9 (99-281) | 0 903 |
| Smoking history | | | | | |
| No | 172 (81.5%) | 175 (82.9%) | 170 (79.8%) | 171 (83.8%) | 0.679 |
| Yes | 39 (18.5%) | 36 (17 1%) | 43 (20 2%) | 33 (16.2%) | |
| Estradiol (pg/mL) | 8.40 (1.6-24.5) | 10.85 (1 4-67 9) | 876 (1 5-34.7) | 7 99 (1.5-49 4) | 0 410 |
| FSH (mlU/mL) | 76 8 (24-136) | 81.5 (40-163) | 72 1 (25-138) | 75 4 (32-13 9) | 0 332 |

E₂, 17β-estradiol,
LNG, levonorgestrel;
FSH, follicle-stimulating hormone.

Of the 293 women randomized to treatment in study 1, 42 withdrew from the study and prematurely discontinued study medication for the following reasons: protocol violations (n=10), adverse events (n=17), lack of efficacy (n=8), withdrawal of consent (n=3), or other (n=4). The ITT population comprised 283 women.

Of the 845 women randomized to treatment in study 2, five never received any study medication, and another eight were excluded from all efficacy analyses. The ITT population therefore consisted of 832 women. During study 2, an additional 392 subjects withdrew prematurely because of adverse events (n=256), protocol violations (n=17), lack of efficacy (n=15), withdrawal of consent (n=39), death (n=2), or other/lost to follow-up (n=63). There were 126 women eligible for the symptom substudy analysis, of whom 122 could be evaluated. Of the 256 women who withdrew because of adverse events, 69 were in the transdermal E₂/LNG 4.4/1.39-mg group, 66 were in the 4.4/2.75-mg group, 67 were in the 4.5/3.75-mg group, and 54 were in the E₂ 4.4-mg group. The most common adverse events leading to treatment withdrawal were vaginal hemorrhage (n=102), application-site reactions (n=71), and breast pain (n=15). Vaginal hemorrhage was defined as any bleeding from the vagina (as might be expected from HRT), not to be confused with severe bleeding (as is evidenced by the low incidence of HCT change). The two deaths in the study were not considered to be treatment related. One woman receiving E₂/LNG 4.4/2.75 mg died of a cardiac arrest, and another woman receiving E₂/LNG 4.5/3.75 mg died of lung cancer with brain metastases.

Hot Flushes

Frequency

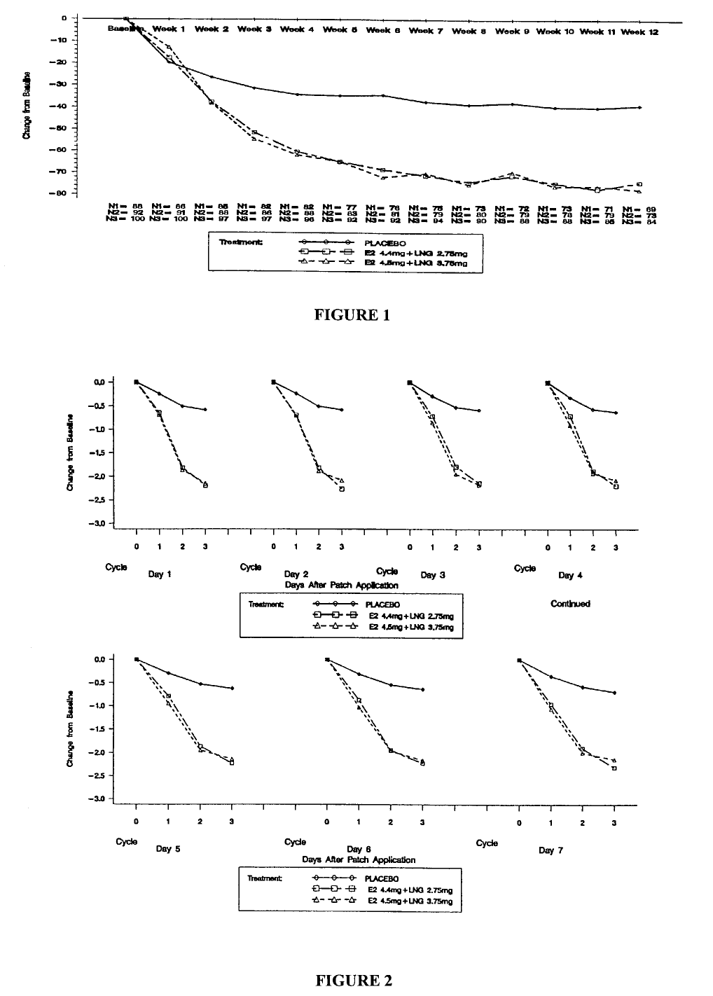

In study 1, a decrease from baseline in the mean weekly hot flush frequency was evident after 1 week of treatment with both doses of transdermal E₂/LNG; a significant difference with both doses was evident versus placebo at the end of week 2 (p≦0.007; FIG. 1). At endpoint, the mean weekly number of hot flushes had decreased by 72.02 from baseline with the E₂/LNG 4.4/2.75-mg dose (p<0.001) and by 68.25 with the 4.5/3.75-mg dose (p<0.001) compared with a decrease of 37.74 with placebo. Significant differences versus placebo in the change from baseline in the mean weekly number of hot flushes were also evident with both E₂/LNG doses at cycles 1, 2, and 3 (p<0.001).

In the symptom substudy of study 2, all three doses of transdermal E₂/LNG and transdermal E₂ reduced the weekly number of hot flushes from baseline at endpoint; there were no significant differences between treatment groups at any time point.

At endpoint in study 1, the mean decrease from baseline in the daily number of hot flushes was 10.13 with transdermal E₂/LNG 4.4/2.75 mg (p<0.001) and 9.32 with the 4.5/3.75-mg dose (p<0.001) compared with placebo (5.14). Respective baseline values were 12.49, 11.83, and 13.04. In study 2 at endpoint, mean decreases in the mean daily number of hot flushes from baseline were 4.58, 5.57, 5.42, and 6.47 with transdermal E₂/LNG 4.4/1.39 mg, 4.4/2.75 mg, 4.5/3.75 mg, and E2 4.4 mg, respectively; there were no significant differences between treatment groups at any time point.

Severity

At endpoint in study 1, a reduction from baseline in maximal hot flush severity of approximately 1.9 to 2.2 was achieved with both doses of transdermal E₂/LNG versus a reduction of approximately 0.5 to 0.6 with placebo. Differences between transdermal E₂/LNG and placebo were statistically significant (p<0.001) at all time points in each cycle, although the effect of treatment became more marked over time (FIG. 2). The median baseline hot flush severity score was three for all treatment groups; hot flush severity improved from severe to mild with both doses of transdermal E₂/LNG after 3 months of treatment, whereas hot flushes in placebo recipients remained moderately severe at endpoint.

Similarly, in the symptom subanalysis of study 2 (118 evaluatable subjects), the maximal hot flush severity decreased in all treatment groups, with no significant differences between groups at any time point.

Urogenital Symptoms

In study 1, both doses of transdermal E₂/LNG significantly reduced the proportion of women experiencing vaginal dryness. At endpoint (final week 1), 80.9% and 80.0% of E₂/LNG 4.4/2.75 mg (n=89; p=0.013) and 4.5/3.75 mg (n=100; p=0.016) recipients, respectively, reported no vaginal dryness compared with 64.8% of placebo recipients (n=88); significant improvements were observed from the second cycle of treatment onward. No other urogenital symptoms (dyspareunia, frequent urination, dysuria, stress incontinence, nocturia) were significantly improved with transdermal E₂/LNG versus placebo at endpoint.

In study 2, the proportion of women experiencing vaginal dryness, dyspareunia, dysuria, stress incontinence, and nocturia was similar in all treatment groups at endpoint. Frequent urination, however, was reported in significantly fewer women receiving transdermal E₂/LNG 4.4/3.75 mg [5/29 (17.2%)] than transdermal E₂ 4.4 mg [16/34 (47.1%) p=0.013] at endpoint.

Endometrial Hyperplasia

Over the 1-year course of study 2, 19 women (12.8%) who received transdermal E₂ 4.4 mg developed endometrial hyperplasia, compared with no women in any of the combined transdermal E₂/LNG groups (Table 3). The differences were significant for each dose of combined E₂/LNG compared with unopposed E₂ (p<0.001). No cases of endometrial cancer occurred during the study.

TABLE 3
Incidence of endometrial hyperplasia in women
with adequate biopsies at any time during 13 × 28-day
treatment cycles: study 2^{a}

| | E₂/LNG mg/day | | | |
|---|---|---|---|---|
| | 0.045/0.015 | 0.045/0.030 | 0.045/0.040 | 0.045/0.0 |
| | (n = 147)^{b} | (n = 138)^{b} | (n = 142)^{b} | (n = 148)^{b} |
| Endometrial | | | | |
| hyperplasia | | | | |
| Yes | 0 | 0 | 0 | 19^{c} (12.8%) |
| No | 147 (100%) | 138 (100%) | 142 (100%) | 129 (87.2%) |

E₂, 17β-estradiol;
LNG, levonorgestrel.
^{a}p value (Fisher's exact test) < 0.001.
^{b}Excludes those who withdrew prematurely and in whom no biopsies were available (n = 213), those with inadequate biopsies (n = 44), and those with endometrial hyperplasia at baseline (n = 1).
^{c}Simple (n = 17) or atypical (n = 2) hyperplasia.

Well-Being

The effects of 13 cycles of treatment on WHQ scores are summarized in Table 4. At endpoint, there were no significant differences between treatment groups in any WHQ subscore (somatic symptoms, depressed mood, vasomotor symptoms, anxiety/fears, sexual functioning, sleep problems, cognitive difficulties, menstrual problems, or attractiveness) or total score. However, all treatment groups showed statistically significant improvement from baseline in vasomotor symptoms, sleep problems, and the total score at all time points, and in sexual function and cognitive difficulties at most time points.

TABLE 4
Mean change (SD) in women's health questionnaire scores and subscores
versus baseline at endpoint (cycle 13): study 2

| | E₂/LNG mg/day | | | | | | | |
|---|---|---|---|---|---|---|---|---|
| Domain | 0.045/0.015 (n = 206)^{a} | | 0.045/0.030 (n = 207)^{a} | | 0.045/0.040 (n = 209)^{a} | | 0.045/0.0 (n = 199)^{a} | |
| Somatic symptoms | 0.10 | (3.12) | 0.27 | (3.59) | 0.28 | (3.27) | −0.16 | (2.85) |
| Depressed mood | 0.03 | (3.09) | 0.27 | (3.55) | 0.17 | (3.00) | 0.11 | (3.05) |
| Vasomotor symptoms | 1.72 | (2.24) | 1.75 | (2.29) | 1.71 | (2.20) | 1.47 | (2.05) |
| Anxiety/fears | −0.04 | (2.28) | 0.40 | (2.50) | 0.30 | (2.09) | 0.03 | (2.03) |
| Sexual functioning | 0.57 | (2.81) | 0.44 | (2.61) | 0.23 | (2.96) | 0.40 | (3.05) |
| Sleep problems | 0.51 | (1.95) | 0.63 | (2.13) | 0.53 | (2.06) | 0.45 | (1.87) |
| Cognitive difficulties | 0.20 | (1.95) | 0.49 | (2.03) | 0.27 | (1.93) | 0.07 | (1.78) |
| Menstrual problems | −0.06 | (3.17) | −0.51 | (3.25) | −0.23 | (3.27) | −0.20 | (3.19) |
| Attractiveness | 0.10 | (1.32) | 0.18 | (1.56) | 0.03 | (1.38) | −0.03 | (1.19) |
| Total score | 3.00 | (12.34) | 3.86 | (14.67) | 3.27 | (12.75) | 2.09 | (11.56) |
| p value (total score) | 0.28 | | 0.28 | | 0.43 | | | |

E₂, 17β-estradiol;
LNG, levonorgestrel.
^{a}Endpoint analysis, which was the final evaluation on medication carried forward.

Tolerability

All adverse events reported at an incidence of ≧2% in study 1 are summarized in Table 5. Application-site reactions, vaginal hemorrhage, and upper respiratory infections were the most common events reported; however, upper respiratory infection was not considered to be drug related in any patient. Application-site reactions led to withdrawal of three (3%) placebo recipients, one (1%) transdermal E₂/LNG 4.4/2.75-mg recipient, and two (2%) 4.5/3.75-mg recipients; vaginal hemorrhage led to withdrawal in zero, two, and two women, respectively. None of the women experienced a serious adverse event.

Similarly, in study 2, the most common adverse events with transdermal E₂/LNG were application-site reactions (31.0% to 44.1%), vaginal hemorrhage, and breast pain. Vaginal hemorrhage (29.4% to 37.1%) and breast pain (16.1% to 22.5%) were more common with combined E₂/LNG than with unopposed E₂ (21.6% and 9.8%, respectively). However, endometrial disorder was more common with E₂ alone (7.8%) than with combined transdermal E₂/LNG (0.5% to 2.3%). Application-site reactions led to withdrawal in 23 (10.8%), 18 (8.5%), 12 (5.6%), and 18 (8.8%) women receiving transdermal E₂/LNG 4.4/1.39 mg, 4.4/2.75 mg, and 4.5/3.75 mg, and E₂ 4.4 mg, respectively. Respective values for vaginal hemorrhage were 26 (12.3%), 26 (12.3%), 32 (15.0%), and 18 (8.8%). Serious events were reported at a similar incidence in all treatment groups, and most were not considered to be treatment related.

TABLE 5
Incidence of all adverse events reported
by ≧2% of subjects: study 1

E₂/LNG mg/day

| | 0.045/0.030 | | 0.045/0.040 | | Placebo | |
|---|---|---|---|---|---|---|
| Adverse event | (n = 96) | | (n = 104) | | (n = 93) | |
| Patients experiencing ≧1 | 67 | (69.8%) | 68 | (65.4%) | 61 | (65.6%) |
| event | | | | | | |
| Events leading to withdrawal | 6 | (6.3%) | 5 | (4.8%) | 6 | (6.5%) |
| Body as a whole | | | | | | |
| Abdominal pain | 4 | (4.2%) | 4 | (3.8%) | 2 | (2.2%) |
| Accidental injury | 3 | (3.1%) | 2 | (1.9%) | 3 | (3.2%) |
| Back pain | 4 | (4.2%) | 2 | (1.9%) | 1 | (1.1%) |
| Digestive | | | | | | |
| Flatulence | 3 | (3.1%) | 4 | (3.8%) | 0 | |
| Metabolic/nutrition | | | | | | |
| Edema | 1 | (1.0%) | 3 | (2.9%) | 2 | (2.2%) |
| Weight gain | 4 | (4.2%) | 4 | (3.8%) | 1 | (1.1%) |
| Nervous system | | | | | | |
| Emotional lability | 2 | (2.1%) | 2 | (2.9%) | 2 | (2.2%) |
| Headache | 7 | (7.3%) | 3 | (1.9%) | 8 | (8.6%) |
| Respiratory | | | | | | |
| Sinusitis | 3 | (3.1%) | 1 | (1.0%) | 2 | (2.2%) |
| Upper respiratory infection | 10 | (10.4%) | 6 | (5.8%) | 7 | (7.5%) |
| Skin | | | | | | |
| Application site | 36 | (37.5%) | 37 | (35.6%) | 39 | (41.9%) |
| Breast pain | 4 | (4.2%) | 8 | (7.7%) | 2 | (2.2%) |
| Rash | 4 | (4.2%) | 2 | (1.9%) | 4 | (4.3%) |
| Urogenital | | | | | | |
| Vaginal hemorrhage | 11 | (11.5%) | 11 | (10.6%) | 0 | |
| Vaginal moniliasis | 3 | (3.1%) | 2 | (1.9%) | 1 | (1.1%) |
| Vaginitis | 3 | (3.1%) | 5 | (4.8%) | 0 | |

E₂, 17β-estradiol;
LNG, levonorgestrel.

Bleeding Patterns

The proportion of women with amenorrhea increased in all treatment groups, according to a cumulative analysis performed over 12 months (Table 6). Likewise, the number of bleeding days decreased over the study period in all E₂/LNG groups; however, at endpoint, the number of bleeding days was significantly greater with transdermal E₂/LNG versus E₂, with the exception of the E₂/LNG 4.4/2.75-mg dose (Table 6). In general, the proportion of patients with any spotting and the number of spotting days was significantly greater with E₂/LNG versus E₂ up to cycle 6, but did not differ significantly between groups thereafter.

TABLE 6
Bleeding patterns at endpoint (cycle 12 or 13): study 2

| | E₂/LNG mg/day | | | | | | | |
|---|---|---|---|---|---|---|---|---|
| Parameter | 0.045/0.015 | | 0.045/0.030 | | 0.045/0.040 | | 0.045/0.0 | |
| Amenorrhea [n (%)]^{a} | (n = 97) | | (n = 96) | | (n = 98) | | (n = 98) | |
| Cycle 3 | 14 | (14.4) | 13 | (13.5) | 14 | (14.3) | 42 | (42.9) |
| Cycle 6 | 19 | (19.6) | 22 | (22.9) | 21 | (21.4) | 44 | (44.9) |
| Cycle 9 | 28 | (28.9) | 31 | (32.3) | 33 | (33.7) | 56 | (57.1) |
| Completers endpoint | 40 | (41.2) | 47 | (49.0) | 52 | (53.1) | 74 | (75.5) |
| Bleeding days [mean (SD)] | (n = 209) | | (n = 209) | | (n = 211) | | (n = 200) | |
| Cycle 3 | 4.75 | (5.63) | 4.29 | (5.79) | 5.18 | (6.28) | 1.07 | (2.88) |
| Cycle 6 | 4.27 | (5.49) | 3.31 | (4.95) | 4.72 | (5.95) | 1.48 | (4.43) |
| Cycle 9 | 3.62 | (5.24) | 2.64 | (4.57) | 4.13 | (5.81) | 1.25 | (3.83) |
| Endpoint | 3.56 | (4.98) | 2.97 | (4.90) | 3.58 | (5.33) | 2.73 | (5.60) |
| p value (endpoint) | <0.001 | | 0.039 | | 0.003 | | | |

E₂, 17β-estradiol;
LNG, levonorgestrel.
^{a}Cumulative analysis to the end of cycle 12 for subjects with 14 or more days of data for each cycle.

Changes in lipid parameters from baseline in study 2 are summarized in Table 7. At endpoint, combined transdermal E₂/LNG was associated with changes in the lipid profile that included significant reduction in total cholesterol and triglyceride levels, reduction in low-density lipoprotein (LDL) levels, reductions in total cholesterol, and minor reduction in high-density lipoprotein (HDL) levels. All changes were significant versus baseline, except for the LDL value of the E₂/LNG 4.4/2.75-mg group. In comparison, unopposed E2 decreased total cholesterol and LDL levels but was associated with increased levels of triglycerides and HDL. However, none of these changes was significantly different from the baseline values. Differences between transdermal E₂/LNG (all doses) and E₂ groups in total cholesterol, triglycerides, and HDL were significant at endpoint, with the exception of total cholesterol values for E₂/LNG 4.4/2.75 mg (Table 7).

TABLE 7
Change in lipid profile versus baseline at endpoint (cycle 13): study 2

| | E₂/LNG mg/day | | | |
|---|---|---|---|---|
| Parameter [mean value (SD)] | 0.045/0.015 | 0.045/0.030 | 0.045/0.040 | 0.045/0.0 |

| | | | | |
|---|---|---|---|---|
| Total cholesterol (mg/dL) | (n = 113) | (n = 105) | (n = 116) | (n = 113) |

| Baseline | 219.1 | (38.09) | 217.6 | (35.19) | 220.7 | (35.55) | 219.4 | (36.65) |
|---|---|---|---|---|---|---|---|---|
| Endpoint | −14.2 | (29.61) | −11.8 | (33.43) | −22.1 | (28.14) | −3.2 | (29.96) |
| p value^{a} | <0.001 | | <0.001 | | <0.001 | | 0.263 | |
| p value^{b} | 0.004 | | 0.109 | | <0.001 | | — | |
| Triglycerides (mg/dL) | (n = 114) | | (n = 106) | | (n = 117) | | (n = 113) | |
| Baseline | 130.0 | (57.17) | 127.0 | (54.25) | 132.8 | (62.64) | 124.1 | (54.33) |
| Endpoint | −24.0 | (48.78) | −13.1 | (55.50) | −26.0 | (51.16) | 1.5 | (53.72) |
| p value^{a} | <0.001 | | 0.017 | | <0.001 | | 0.772 | |
| p value^{b} | <0.001 | | 0.048 | | <0.001 | | — | |
| HDL (mg/dL) | (n = 112) | | (n = 104) | | (n = 116) | | (n = 112) | |
| Baseline | 56.0 | (14.30) | 56.3 | (13.52) | 56.5 | (14.93) | 57.6 | (14.81) |
| Endpoint | −2.4 | (8.17) | −5.2 | (9.53) | −5.4 | (10.48) | 0.9 | (9.29) |
| p value^{a} | 0.002 | | <0.001 | | <0.001 | | 0.284 | |
| p value^{b} | 0.013 | | <0.001 | | <0.001 | | — | |
| LDL (mg/dL) | (n = 112) | | (n = 104) | | (n = 116) | | (n = 110) | |
| Baseline | 137.0 | (35.60) | 136.2 | (33.61) | 137.9 | (33.15) | 136.9 | (33.94) |
| Endpoint | −7.7 | (26.26) | −4.1 | (27.46) | −11.7 | (22.80) | −4.4 | (24.30) |
| p value^{a} | 0.002 | | 0.133 | | <0.001 | | 0.060 | |
| p value^{b} | 0.316 | | 0.545 | | 0.090 | | — | |

E₂, 17β-estradiol;
LNG, levonorgestrel,
^{a}p value for the change from screening within a treatment group (p < 0.05)
^{b}p value for comparison with unopposed estradiol (p < 0.05)

These data are consistent with those from study 1, which reported statistically significant decreases in total cholesterol, HDL, and LDL levels with E₂/LNG 4.4/2.75 mg and 4.5/3.75 mg versus placebo after three cycles of treatment (p<0.001).

Pap Smears

In study 1, none of the 263 women who had Pap smears had changes that were considered to be clinically significant. In study 2, of 663 women who had Pap smears, three (1.8%) in the E₂ 4.4-mg group, zero in the E₂/LNG 4.4/1.39-mg group, one (0.6%) in the E₂/LNG 4.4/2.75-mg group, and one (0.6%) in the E₂/LNG 4.5/3.75-mg group who had benign cellular findings at screening showed epithelial cell abnormalities at endpoint.

Other Tolerability Data

At endpoint, no statistically significant differences in body weight, heart rates, and diastolic and systolic blood pressure versus baseline were observed in any treatment group in either study. Abnormal laboratory test findings (hematology, blood chemistry, and urinalysis) considered to be adverse events were reported in 0.1% to 0.5% of subjects, with a similar pattern of events between treatment groups.

The entire disclosures of all applications, patents and publications, cited herein are incorporated by reference herein including Shulman et al., Menopause: The Journal of The North American Menopause Society, 9(3): 195-207 (2002) and Shulman, Effects of Continuous Once-a-Week Transdermal 17β-Estradiol/Levonorgestrel (Climara Pro) Versus Transdermal Estradiol on Lipids in Postmenopausal Women in 1-Year Randomized Double Blind Trial (Poster Presentation at the North American Menopause Society meeting (October 2001).

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

What is claimed is:

1. A method for reducing triglyceride levels and achieving hormone replacement therapy in a patient comprising continuously transdermally administering a composition comprising 17β-estradiol and levonorgestrel.

2. A method for reducing triglyceride levels and achieving hormone replacement therapy in a patient comprising continuously transdermally administering an essentially constant therapeutically effective amount of 17β-estradiol and levonorgestrel in a pharmaceutically acceptable carrier.

3. The method of claim 2, wherein said 17β-estradiol and levonorgestrel are formulated for transdermal delivery as a single patch.

4. The method of claim 2, wherein said 17β-estradiol and levonorgestrel are each formulated for transdermal delivery as separate patches and administered simultaneously.

5. The method of claim 1, wherein said 17β-estradiol is present in an amount ranging from about 3 mg to about 6 mg.

6. The method of claim 5, wherein said 17β-estradiol is present in an amount ranging from about 4 mg to about 5 mg.

7. The method of claim 6, wherein said 17β-estradiol is present in an amount ranging from about 4.4 mg to about 4.5 mg.

8. The method of claim 1, wherein said levonorgestrel is present in an amount ranging from about 1 mg to about 5 mg.

9. The method of claim 8, wherein said levonorgestrel is present in an amount ranging from about 1 mg to about 4 mg.

10. The method of claim 9, wherein said levonorgestrel is present in an amount ranging from about 1.39 mg to about 3.75 mg.

11. The method of claim 1, wherein said 17β-estradiol has a delivery rate ranging from abut 0.025 mg/day to about 0.1 mg/day and said levonorgestrel has a delivery rate ranging from about 0.015 mg/day to about 0.040 mg/day.

12. The method of claim 11, wherein said 17β-estradiol has a delivery rate ranging from abut 0.04 mg/day to about 0.05 mg/day and said levonorgestrel has a delivery rate ranging from about 0.015 mg/day to about 0.040 mg/day.

13. The method of claim 1, wherein said 17β-estradiol has a delivery rate of about 0.045 mg/day and said levonorgestrel has a delivery rate of about 0.015 mg/day.

14. The method of claim 1, wherein said 17β-estradiol has a delivery rate of about 0.045 mg/day and said levonorgestrel has a delivery rate of about 0.030 mg/day.

15. The method of claim 1, wherein said 17β-estradiol has a delivery rate of about 0.045 mg/day and said levonorgestrel has a delivery rate of about 0.040 mg/day.

16. The method of claim 2, wherein said 17β-estradiol is present in an amount ranging from about 3 mg to about 6 mg.

17. The method of claim 16, wherein said 17β-estradiol is present in an amount ranging from about 4 mg to about 5 mg.

18. The method of claim 17, wherein said 17β-estradiol is present in an amount ranging from about 4.4 mg to about 4.5 mg.

19. The method of claim 2, wherein said levonorgestrel is present in an amount ranging from about 1 mg to about 5 mg.

20. The method of claim 19, wherein said levonorgestrel is present in an amount ranging from about 1 mg to about 4 mg.

21. The method of claim 20, wherein said levonorgestrel is present in an amount ranging from about 1.39 mg to about 3.75 mg.

22. The method of claim 2, wherein said 17β-estradiol has a delivery rate ranging from abut 0.025 mg/day to about 0.1 mg/day and said levonorgestrel has a delivery rate ranging from about 0.015 mg/day to about 0.040 mg/day.

23. The method of claim 22, wherein said 17β-estradiol has a delivery rate ranging from abut 0.04 mg/day to about 0.05 mg/day and said levonorgestrel has a delivery rate ranging from about 0.015 mg/day to about 0.040 mg/day.

24. The method of claim 2, wherein said 17β-estradiol has a delivery rate of about 0.045 mg/day and said levonorgestrel has a delivery rate of about 0.015 mg/day.

25. The method of claim 2, wherein said 17β-estradiol has a delivery rate of about 0.045 mg/day and said levonorgestrel has a delivery rate of about 0.030 mg/day.

26. The method of claim 2, wherein said 17β-estradiol has a delivery rate of about 0.045 mg/day and said levonorgestrel has a delivery rate of about 0.040 mg/day.

27. A transdermal patch comprising 17β-estradiol and LNG in sufficient amounts to effect a delivery rate ranging from abut 0.025 mg/day to about 0.1 mg/day and from about 0.015 mg/day to about 0.040 mg/day, respectively.

28. A transdermal patch comprising 17β-estradiol and LNG in sufficient amounts to effect a delivery rate ranging from abut 0.04 mg/day to about 0.05 mg/day and from about 0.015 mg/day to about 0.040 mg/day, respectively.

29. A transdermal patch comprising 17β-estradiol and LNG in sufficient amounts to effect a delivery rate of about 0.045 mg/day and about 0.015 mg/day, respectively.

30. A transdermal patch comprising 17β-estradiol and LNG in sufficient amounts to effect a delivery rate of about 0.045 mg/day and about 0.030 mg/day, respectively.

31. A transdermal patch comprising 17β-estradiol and LNG in sufficient amounts to effect a delivery rate of about 0.045 mg/day and about 0.040 mg/day, respectively.
